(19) United States
(12) Patent Application Publication
(10) Pub. No.: US 2015/0290014 A1
(43) Pub. Date: Oct. 15, 2015

(54) DEVICE FOR HOLDING THE PATELLA AND KNEE ORTHOSIS

(71) Applicant: GIBAUD, Saint Etienne (FR)

(72) Inventors: Gerard Anglada, Saint Etienne (FR); Frederic Girard, Paris (FR)

(21) Appl. No.: 14/439,709

(22) PCT Filed: Sep. 26, 2013
(86) PCT No.: PCT/FR2013/052264

§ 371 Date: Apr. 30, 2015

(30) Foreign Application Priority Data
Oct. 30, 2012 (FR) 12/60363

(51) Int. Cl. A61F 5/01

(52) U.S. Cl. CPC A61F 5/0125

(57) ABSTRACT

An orthosis includes a device for holding the knee cap and arranged to press against the knee by a part consisting of an elastic material surrounding the knee. The holding device includes an annular body having a central oval opening for receiving the knee cap; and separate supporting bodies that protrude outwardly from the periphery of the body. Two adjacent supporting bodies are separated by a gap when the holding device in a non-constrained state. The holding device is substantially flat in the non-constrained state and non-deformable in a plane. The holding device is designed so as to be able to be deformed into a domed position, hugging the shape of the bent knee. In this position, the supporting bodies are butt jointedand the holding device forms a shell enveloping the knee.

The present invention relates to a device for holding the patella and a knee orthosis comprising such a holding device.

The invention is more particularly intended for people who are subject to dislocation or sub-dislocation of the patella. The dislocation corresponds to the exit of the patella from the housing of the femur in which it is normally received, towards the outside, whereas the sub-dislocation is a bad-positioning of the patella without there being a real dislocation of the latter. The dislocation of the patella constitutes a painful and shocking accident, translated by the deformation of the knee and the blocking thereof.

The orthoses used for dislocation and sub-dislocation of the patella are set up either after a surgical procedure, or in substitution of the latter, or in a preventive manner. Wearing such orthoses is substantially carried out while practicing sport.

In a known manner, such a knee orthosis includes a part made of an elastic material intended to surround the knee. In addition, in order to prevent the dislocation of the patella, it may be provided an insert in arc of circle which is placed on the outer side of the patella, with its concavity turned towards the patella. This insert thus forms a stop aiming to prevent or at least limit, the displacement of the patella outwards. It is generally associated to straps assembled to the part made of an elastic material and on which the user pulls in the direction opposite the dislocation, that is to say towards the median plane. However, this action may quickly become disturbing for the user, particularly during excess traction.

Moreover, despite the traction exerted by the straps, the stop effect provided by the insert may reveal to be insufficient for holding the patella in place, and thus in particular during the bending of the knee, where the risk of dislocation is the most important.

The present invention aims to overcome the aforementioned drawbacks.

To this end, and according to a first aspect, the invention relates to a device for holding the patella intended to be placed against the knee of a person, this device comprising:

a substantially annular body having a central opening which is intended to receive the patella and which has a substantially oval edge;

a plurality of support members distinct from one another, protruding outwards from the periphery of the body, two adjacent support members being separated by a slit in the unconstrained state of the holding device.

The holding device is substantially planar in the unconstrained state; it is substantially non deformable in this plane; in addition, it is designed to be able to be deformed into a convex position espousing the shape of the knee when bending, a convex or substantially domed position in which the support members are substantially contiguous, the holding device then substantially forming a cup enveloping the knee.

Thus, the invention provides a holding device which not only forms a stop but in a more complete manner a means for enclosing the patella aiming to hold it in its normal position in the housing of the femur. It results an improved efficiency of the holding device.

In addition, this enclosing is even more important when the knee is bent, since the holding device then forms a cup, for example substantially hemispherical, formed by the support members which have become closer together making the slits disappear. The bent knee is then substantially contained in this cup. This is particularly advantageous as when the knee is bent the risk of dislocation is the greatest.

With the holding device according to the invention, the support members are always in contact with the knee whatever the degree of flexion of the latter. The holding device, which is substantially planar in the unconstrained state, may be suited to a non planar surface and the shape thereof may evolve depending on the movements of the person (in particular flexion and extension), thus guaranteeing a substantially permanent peripheral contact around the patella.

The skin adhesion of the material constituting the holding device can complete the mechanical action of containment of the patella.

Furthermore, by virtue of its ring shape with a central opening and its distinct peripheral support members, the holding device is simple and intuitive to position on the knee, and is substantially suitable for all morphotypes (normal axis, genu valgum, genu varum).

The holding device may typically be tightened against the knee by means of a part made of an elastic material surrounding the knee. In such a manner, the efficient holding of the patella is not essentially obtained by the traction on straps. Thus, the risk of an excessive tightening, and the resulting discomfort, are avoided. This device and the knee orthosis comprising it may hence be used for a long time in particular during sports.

In practice, so that the holding device is not substantially deformable in the plane it occupies in the unconstrained state, the material constituting the device and/or the geometry thereof may be appropriately chosen. Particularly, it may be provided that the edge of the opening of the annular body be deprived of indentations, that is to say, in other words, that the support members do not have a cut-out arranged on the inner side (side turned towards the opening).

By “substantially non deformable”, is meant that the holding device substantially keeps its shape in the normal conditions of use. Particularly, in the normal conditions of use, the holding device cannot be significantly stretched along a direction contained in said plane. A slight extension of the device may however occur, in particular when the person bends his/her knee, the elongation of the device in a direction not exceeding 10%, and preferably not exceeding 5%.

Nevertheless, it is not excluded that the holding device may become substantially deformed as a result of a traction exerted with a sufficiently strong force, but not corresponding to the constraints to which the device is subjected to in the normal conditions of use.

Thus, the holding device has a certain stiffness allowing it to ensure a satisfactory enclosing of the patella. The holding device also has a certain flexibility, due to the presence of the support members separated by slits, allowing a deformation into a convex shape. It may be understood that the slits must be sufficiently wide to allow the device to take this convex shape, but sufficiently fine to disappear substantially when the knee is bent, for example at around 90°, the support members hence being substantially joined.

According to a possible embodiment, the support members are distributed substantially regularly over substantially the entire periphery of the body. Thus, a large support surface and a balancing of the pressure around the patella is obtained.

The number N of support members may be such that 5≦N≦10, and preferably such that 6≦N≦8. For example, one may have six or seven support members.

The holding device may have a plane of symmetry which, in position of use and when the person equipped with said holding device is standing, is substantially parallel with the median plane of the person- that is to say in a sagittal plane. An advantage of this disposition is that the holding device may be used indifferently on a right knee or a left knee. Another advantage resides in the fact that the holding device does not form a stop for the patella on a privileged side, but ensures a symmetrical enclosing allowing to provide an even more efficient holding in position. Moreover, the symmetry allows improving the centering of the device, as well as the ease of set up thereof

It may be provided that the holding device further comprises a lower support member arranged to be able to press against the anterior tibial tuberosity of the person equipped with said holding device. This allows for the taking care of the Osgood-Schlatter disease and the teno-periostitis of the patellar tendon. The lower support member may be symmetrical with respect to the aforementioned plane, and the holding device may further include six other support members.

The holding device may be fixed on a positioning member designed to be assembled, removeably and displaceably, on a part made of an elastic material intended to surround the knee.

According to a second aspect, the invention relates to a knee orthosis comprising a holding device such as mentioned above and a part made of an elastic material intended to surround the knee and press the holding device against the knee.

The orthosis may further comprise a positioning member on which the holding device is fixed, the positioning member being designed for being able to be held by the user when the orthosis is in the position of use and for being able to be displaced with respect to the part made of an elastic material so that the holding device be positioned correctly around the patella, the positioning member further including removable assembling means on the part made of an elastic material in the appropriate position.

According to a possible embodiment, the orthosis further comprises at least one V-shaped strap which has:

a tip fixed to the part made of an elastic material on a first side of the knee, the tip being directed opposite the knee;

a branch extending above the knee and another branch extending below the knee, the free end of the branches being removeably fixed on the part made of an elastic material.

Advantageously, the orthosis may comprise two V-shaped straps which, in position of use, are disposed in a substantially symmetrical manner on either side of the knee, a strap having:

a tip fixed to the part made of an elastic material on a first side of the knee, the tip being directed opposite the knee;

a branch extending above the knee and another branch extending below the knee, the free end of the branches being removeably fixed on the part made of an elastic material.

In as far as the enclosing of the patella is first obtained by the holding device, which is not connected to the straps, the wearer of the orthosis can adjust the traction on the straps according to his/her preferences without it impacting negatively on the efficiency of the hold of the patella. The invention thus has an important advantage with respect to the orthoses of the prior art in which the quality of the hold of the patella is directly connected to the intensity of the traction on the straps.

The two straps may be arranged so that, in position of use, they intersect, under the knee, substantially on the lower support member of the holding device when the latter is present. Thus, the two branches of the straps which pass under the knee reinforce the compression of the holding device on the anterior tibial tuberosity of the person equipped with said holding device.

At least one strap may include, in the vicinity of the tip thereof, a bow of which the concavity is directed towards the knee, in position of use. This bow contributes to holding the patella and, in this case where each strap comprises a bow, the latter constitute a complement of enclosing of the patella.

Furthermore, the orthosis may comprise, at least on one side of the leg equipped with the orthosis, substantially in the frontal plane in position of use, two bars articulated together substantially at the articulation of the knee, the amplitude of the articulation movement being blocked, in the direction of the extension, at a flexion angle of the order of 5°. Thus, it is prevented that the knee is placed in hyperflexing (recurvatum) position of the knee, which would increase the risk of dislocation of the patella.

It is now described, by way of non limiting example, a possible embodiment of the invention, with reference to the accompanying figures:

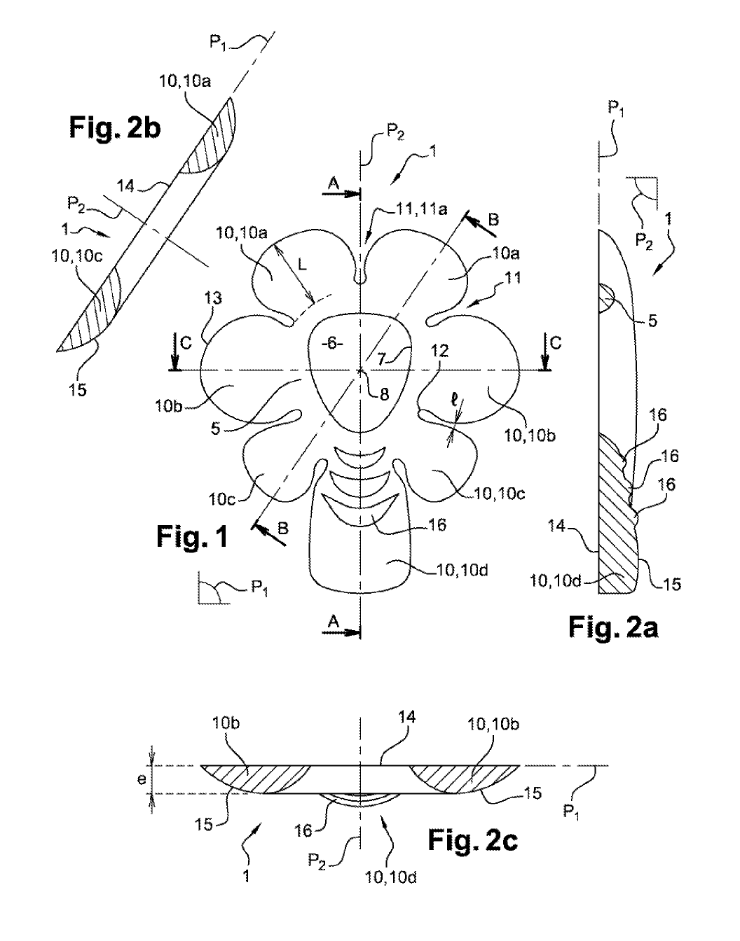

FIG. 1 is a view of a holding device according to the invention, in the unconstrained state;

FIGS. 2a, 2b, 2c are sectional views of the holding device of FIG. 1, respectively according to lines AA, BB and CC;

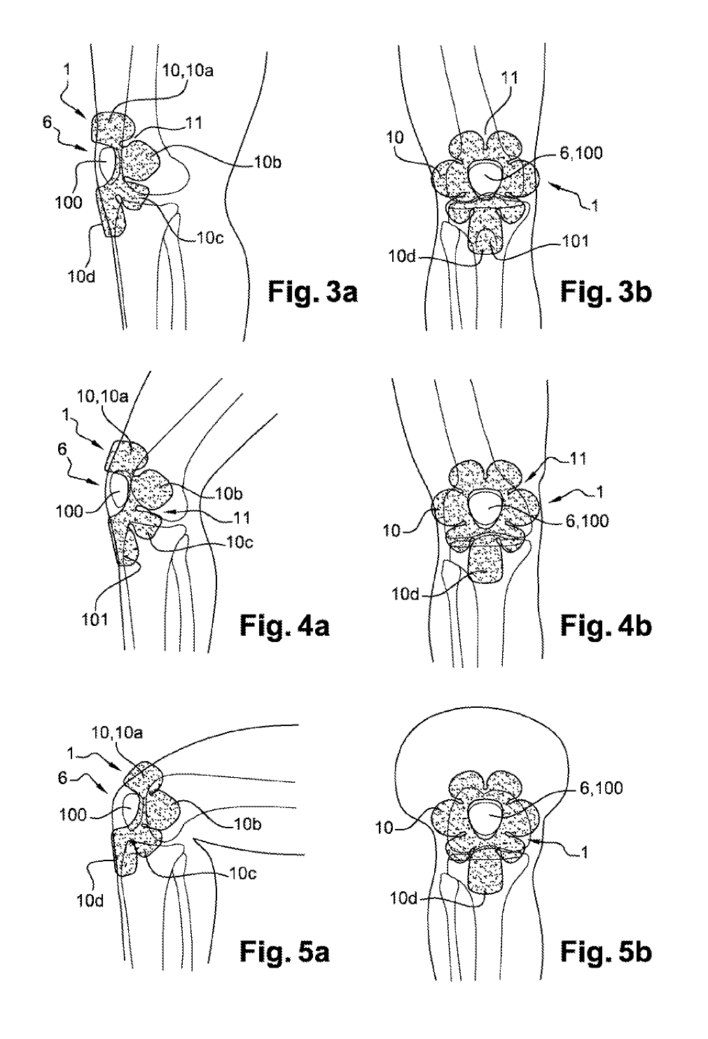

FIGS. 3a and 3b show the holding device positioned on the knee of a person standing in extension, respectively in profile and from the front;

FIGS. 4a and 4b are views similar to FIGS. 3a and 3b, when the knee is bent at an angle ranging between around 30 and 45°;

FIGS. 5a and 5b are views similar to FIGS. 3a and 3bn when the knee is bent at an angle of the order of 90°;

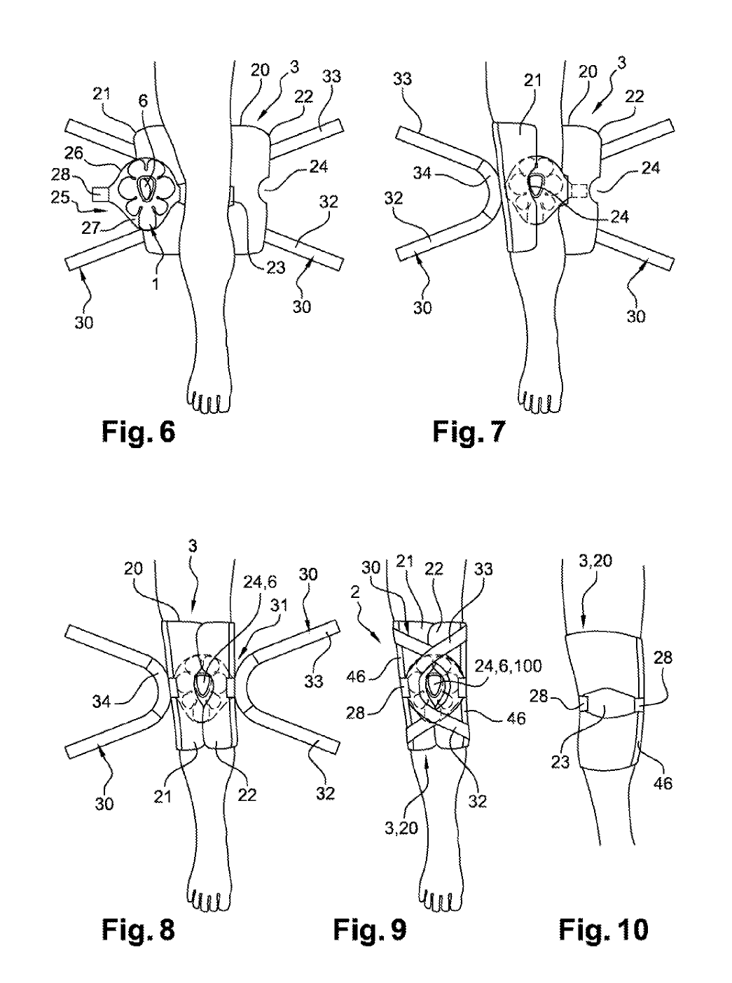

FIGS. 6 to 8 illustrate successive steps of placing a knee orthosis including the holding device of FIG. 1;

FIGS. 9 and 10 show this orthosis in position of use on the knee of a person respectively from the front and from the back;

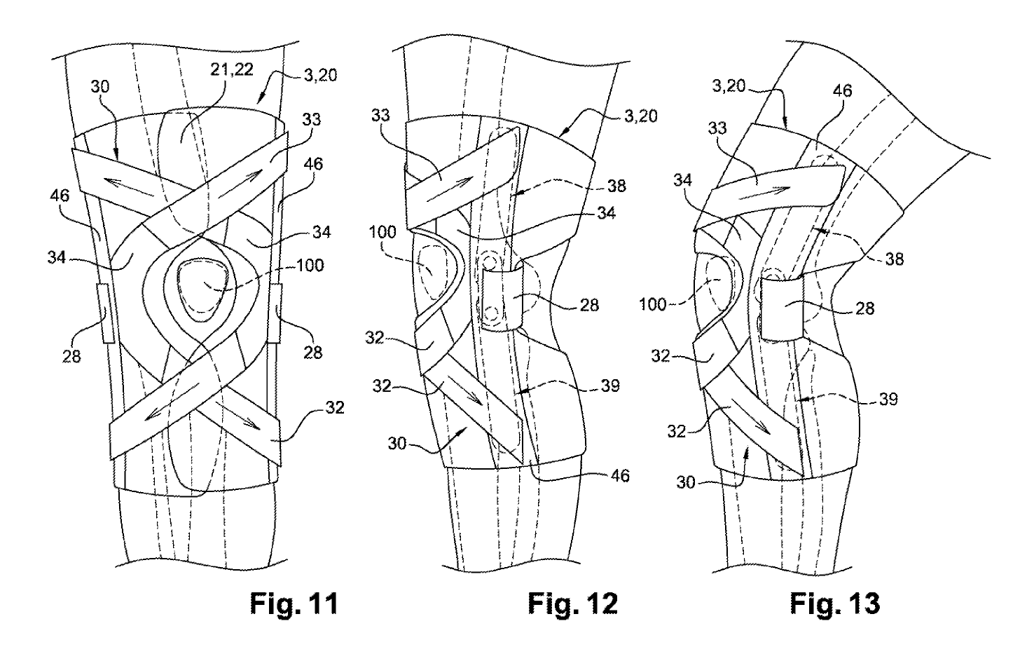

FIGS. 11 to 13 show the effect of the straps of the orthosis on a leg seen respectively from the front and in profile with the knee slightly bent, and in profile with the knee bent;

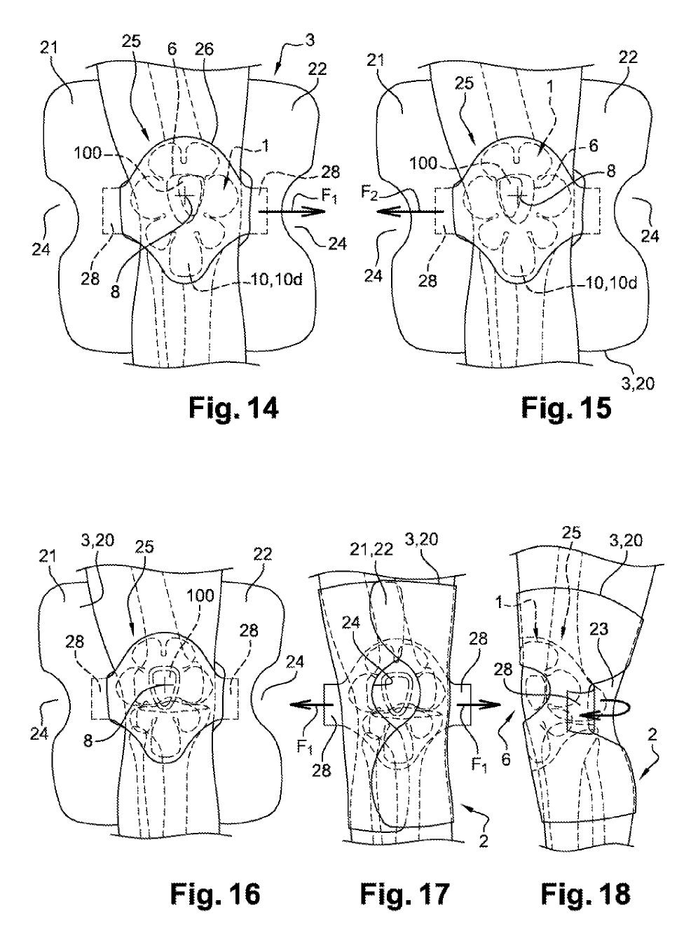

FIGS. 14 and 15 are front views of a knee showing the holding device fixed on a positioning member and the possible displacements of this assembly;

FIGS. 16 to 18 show successive steps of placing the orthosis and more specifically of the assembly comprising the holding device and the positioning member;

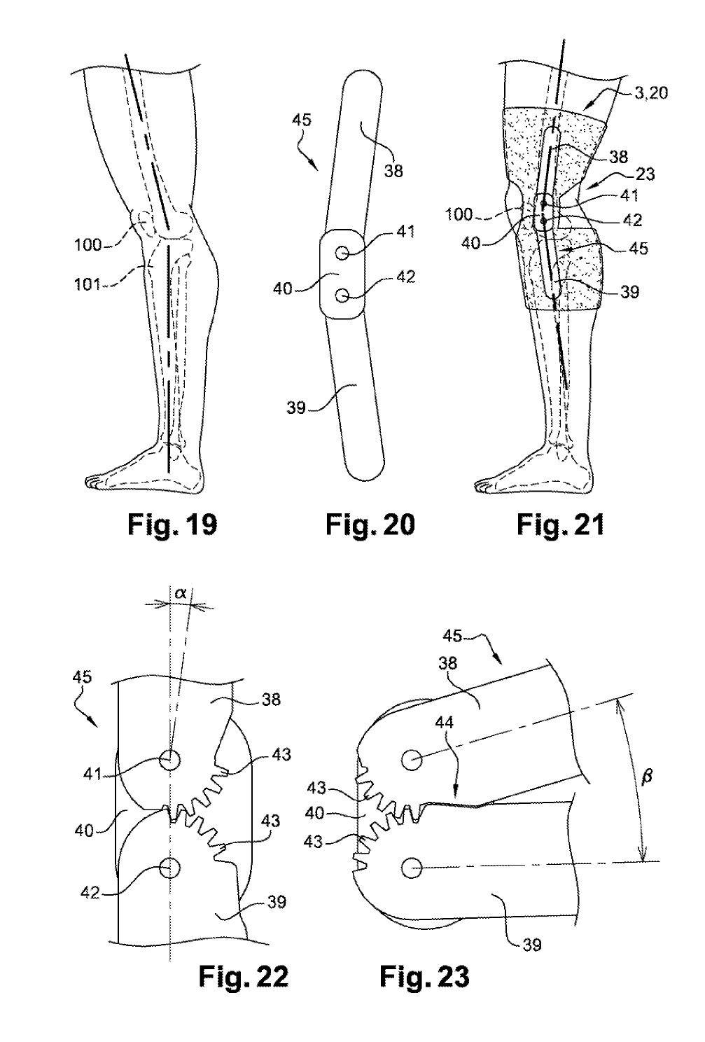

FIG. 19 is a profile view of a leg of a person with the knee in hyperflexing position;

FIG. 20 is a view of two articulated bars able to equip the knee orthosis according to the invention;

FIG. 21 is a profile view of a leg of a person equipped with the orthosis provided with such bars;

FIGS. 22 and 23 schematically show the articulation between the two bars.

FIG. 1 represents a holding device 1 of the patella 100, which is intended to be placed against the knee of a person. More generally, the holding device 1 belongs to a knee orthosis 2 which also comprises a part made of an elastic material 3 intended to surround the knee and press the holding device 1 against the knee.

The holding device 1 may be produced in one single piece, for example by molding. It is preferably produced in a material which imparts it with a certain stiffness, so that it may fulfill the role of holding the patella, but also allowing it to be deformed into a convex or substantially domed position as it is described hereinafter. Furthermore, the material constituting the holding device 1 preferably has a certain skin adhesion preventing or avoiding the sliding thereof with respect to the knee. For example, the holding device 1 may be produced in silicone, in particular in silicone gel with a high viscosity.

The holding device 1 comprises a substantially annular body 5 which has a central opening 6 of which the edge 7 is substantially oval. The opening 6 may in particular be ovoid, the widest portion being located in the upper part in position of use, or even round. The body 5 and the opening 6 have a center 8.

The holding device 1 also comprises a plurality of support members 10 which are distinct from each other and which protrude outwards from the periphery of the body 5, that is to say opposite the opening 6.

In the unconstrained state, the holding device 1 is substantially planar and extends in parallel with a plane P1. The thickness e of the device is for example of the order of 5 to 8 mm (see FIG. 2c).

By “unconstrained state”, is meant the position in which the holding device 1 is in when it is placed on a support such as a table, without being used. The “position of use” designates the position in which the holding device 1 is when it is in place on the knee of a person.

In the unconstrained state, two adjacent support members 10 are separated by a slit 11.

The ends 12 of the slits 11 are preferably located on a substantially oval imaginary line defining the peripheral edge of the body 5. The slits 11 have dimensions allowing to impart the holding device 1 with a sufficient hold for retaining the patella but also allowing the holding device 1 to become deformed into a convex shape. By way of example, the width 1 of the slits 11 may be of the order of 2 to 5 mm.

The holding device 1 here has a symmetry plane P2 which, in position of use and when the person equipped with said holding device 1 is standing, is substantially parallel with the median plane of the person. The plane P2 is orthogonal to the plane P1.

In the represented embodiment, the holding device 1 comprises seven support members 10 distributed substantially regularly over the periphery of the body 5, that is to say each substantially occupying an angular sector near 50° (360°/7). Thus one has:

two upper adjacent support members 10a, arranged on either side of an upper slit 11 a located in the plane P2;

two lateral support members 10b extending along a direction which is substantially orthogonal to the plane P2 in opposite directions;

two low support members 10c;

and a lower support member 10d extending towards the bottom along the direction of intersection of the planes P1 and P2—substantially corresponding to the vertical direction in the position of use.

The lower support member 10d is arranged to be able to bear on the anterior tibial tuberosity 101 of the person equipped with said holding device 1 (see FIGS. 4a and 19).

The support members 10 may have the shape of lobes having a base portion connected to the body 5, a median portion widened with respect to their base portion and an extreme portion having a rounded or flattened free edge 13.

The length L of a support member 10 between the periphery of the body 5—that is to say the end 12 of a corresponding slit 11—and the free end thereof may be of the order of 20 to 30 mm. The lower support member 10d may have a greater length, of the order of 40 to 60 mm.

According to a possible embodiment, and as can be seen on FIGS. 2a, 2b and 2c, the holding device 1 includes a substantially planar external face 14, in the unconstrained state, and the support members 10 have a face 15 turned inwards—that is to say towards the person equipped with the holding device 1 in position of use—which is convex.

The internal face 15 of the holding device 1—that is to say turned towards the person equipped with the holding device in position of use—may include at least one protuberance 16 aiming to prevent sliding on the skin. In the embodiment represented, the holding device comprises protuberances 16 in the shape of brackets. More specifically, the holding device 1 comprises on the lower support member 10d three protuberances 16 in the shape of brackets the concavity of which is directed upwards, and which are disposed below each other.

The holding device 1 is substantially non deformable in the plane P1 which it occupies in the unconstrained state. However, the holding device 1 is designed so as to be deformed into a convex position espousing the shape of the bent knee, a convex or substantially domed position in which the support members 10 are substantially contiguous, the holding device 1 thereby substantially forming a cup enveloping the knee.

The progressive deformation of the holding device 1 into the convex position thereof, according to the bending of the knee, is illustrated on FIGS. 3a to 5b.

The holding device 1 is intended to be placed against the knee of a person, the patella 100 being received in the opening 6. The support members 10 are in contact with the area peripheral to the patella, the lower support member 10d being more specifically located facing the anterior tibial tuberosity 101.

When the person equipped with the holding device 1 is standing, with the knee extended (FIGS. 3a and 3b), the opening 6 contains the patella 100, and the support members 10 distributed around the opening 6 fill the anatomical vacuities thanks to the mobility and low density thereof. This position of the person corresponds to the start of the risk of dislocation of the patella.

In the squatting position, the knee being bent at an angle ranging between around 30° and 45°, the risk of dislocation of the patella is greater. Such as illustrated on FIGS. 4a and 4b, in this position, the support members 10 of the holding device 1 have become closer thanks to their mobility and slits 11 which separate them. The holding device 1 has started to become contained into a convex cup position.

When in the sitting position, with the knee bent at around 90°, there is a significant risk of dislocation of the patella. As illustrated on FIGS. 5a and 5b, in this position, the support members 10 of the holding device 1 are in contact and no longer spaced apart by the slits 11. Then, they form a cup which contains the patella.

Thus, thanks to the invention, the higher the risk of dislocation, the more the holding device 1 contains the patella.

Concretely, the holding device 1 is held and pressed against the knee by the part made of an elastic material 3 which will now be described more specifically.

The portion made of an elastic material 3 is intended to envelop the knee while resting on the leg and the thigh. For example, it is made of an extensible three-dimensional meshed fabric. It may have a plane of symmetry which in position of use and when the person equipped with said holding device 1 is standing, is substantially parallel with the median plane of the person, and is substantially coincident with the plane P2.

According to a possible embodiment, illustrated on the figures, the part made of an elastic material 3 comprises a strip 20 having lateral extreme parts 21, 22 provided with complementary hooking means, so that the strip 20 may be closed and hooked around itself at the anterior part of the knee. Typically, these hooking means may be of the Velcro® type.

Thanks to this full anterior aperture, the placing of the strip is easier, in particular in comparison with a part made of an elastic material shaped like a sleeve which the user must put on. In addition, this allows adjusting the position and setting the pressure exerted on the knee.

The strip 20 may include a substantially central aperture 23 which will be located, when in the position of use, behind the knee, thereby facilitating flexion while preventing the formation of unpleasant folds. In addition, each of the lateral extreme parts 21, 22 of the strip 20 may include a cut-out 24. Once the strip 20 is wound around the knee, the two cut-outs 24 form an aperture for the patella.

The orthosis 2 may further comprise a positioning member 25 on which the holding device 1 is fixed. For example, the positioning member 25 consists of a fabric on which the holding device 1 is molded, in such a manner as to allow securing these two members together and form an assembly. The face of the holding device 1 which is in contact with the positioning member 25 is its external face 14, of which the flatness is then due to the fixing method on the positioning member 25.

The positioning member 25 is designed so as to be assembled, removeably and displaceably, on the part made of an elastic material 3. More specifically, the positioning member 25 is designed so as to be held by the user when the orthosis 2 is in the position of use and to be displaced with respect to the part made of an elastic material 3 in order to properly position the holding device 1 around the patella 100. In addition, the positioning member 25 includes removable assembly means on the part made of an elastic material 3 in the appropriate position. Typically, these assembly means may be of Velcro® type.

According to a possible embodiment, as illustrated in particular on FIGS. 6 and 14, the positioning member 25 has a central portion 26 on the internal face 27 of which the holding device 1 is fixed, as well as two lateral gripping tabs 28 each of which being capable of passing through a lateral lumen arranged in the part made of an elastic material 3 and which are fitted with means for assembly with said part made of an elastic material 3 (for example, the tabs 28 comprise a hook-and-loop fastening fabric). The central portion 26 has, for example, a substantially oval shape corresponding to the envelope of the holding device 1. As regards the lumens, they may be constituted by the lateral ends of the aperture 23 arranged in the part made of an elastic material 3.

The orthosis 2 may further comprise at least one, inextensible or elastic, V-shaped strap 30. In the embodiment represented, the orthosis 2 comprises two V-shaped straps 30, each strap comprising one tip 31 and two branches 32, 33. Equipping the orthosis 2 with two symmetrically disposed straps 30 allows having one single type of orthosis, whether it be for the right leg or for the left leg.

The tip 31 of a strap 30 is fixed to the part made of an elastic material 3 in the vicinity of the first lateral extreme portion 21, with the concavity turned opposite to the strip 20, the two branches 32, 33 extending downwards and upwards, on either side of the cut-out 24. For example, the concavity of the tip 31 substantially surrounds the cut-out 24. The tip 31 of the other strap 30 is fixed to the part made of an elastic material 3 in the vicinity of the second lateral extreme part 22 and it is arranged substantially symmetrically to the first strap with respect to the plane of symmetry of the part made of an elastic material 3.

Thus, when in the position of use, the two straps 30 are disposed in a substantially symmetrical manner on either side of the knee. Hence, each strap 30 has:

a tip 31 fixed to the part made of an elastic material 3 on a first side of the knee, the tip 31 being directed opposite to the knee ;

a branch 33 extending above the knee and another branch 32 extending below the knee.

The free end of the branches 32, 33 is removeably fixed on the part made of an elastic material 3. Typically, the fixing may be obtained by a Velcro® system, the straps 30 being made for example of a hook-and-loop fastening fabric.

At least one strap, and preferably both straps 30 when the orthosis 2 is equipped with two straps, comprises, in the vicinity of its tip 31, a bow 34 the concavity of which is directed towards the knee, when in the position of use.

It is now referred to FIGS. 6 to 8 which illustrate the placing of the orthosis 2 on the knee of a person.

The assembly formed by the positioning member 25 and the holding device 1 is assembled on the strip 20. In practice, this assembly may be performed at one of the two lateral tabs 28, inserted in the aperture 23, the other tab 28 remaining free at this stage. Said assembly is placed so that, once the orthosis is in place, it is the holding device 1 which comes in contact with the knee and not the positioning member 25. The strip 20 thereby equipped is placed behind the knee (FIG. 6).

Then, the user starts folding the strip 20 over the anterior face of his/her knee, for example starting with the extreme portion 21 on which the holding device 1, fixed to the positioning member 25, is associated (FIG. 7).

The user finishes enveloping the strip 20 around his/her knee, while making sure to pass the tab 28 of the positioning member, which until then has remained free, through the aperture 23. Then, he/she hooks the extreme parts 21, 22 of the strip 20 on top of each other (FIG. 8), and assembles said tab 28 to the strip 20. The two cut-outs 24 then form an aperture facing the opening 6 arranged in the holding device 1, facing the patella.

The fact of pulling the tabs 28 forward in order to hook them to the outside of the part made of an elastic material 3 tends to bend the knee slightly, thus contributing to reduce the risk of dislocation of the patella.

The user then puts the straps 30 in place. To this end, after having exerted the desired traction, he/she fixes the free ends of each branch 32, 33 on the part made of an elastic material 3. Preferably, the free end of a branch 32, 33 of a strap 30 is substantially fixed on the side opposite to the side where the tip 31 of the same strap 30 is fixed. Thus, a strap 30 does not perform an entire revolution around the leg, but substantially a half-revolution. This avoids the effects of a tourniquet, especially as the strap 30 is not disposed in a circular manner around the thigh or the leg, but in an oblique manner.

When in the position of use, the two bows 34 surround the patella, thereby contributing to enclosing it. In addition, the two straps 30 may be advantageously arranged so that the two lower branches 32 intersect below the knee, substantially on the lower support member 10d of the holding device 1, in order to reinforce the support in this area. This support contributes to relieve the painful cases of Osgood Schlatter.

FIGS. 9 and 10 respectively show a front and back view of the orthosis 2 in the position of use. The holding device 1 is then tightened between the knee and the part made of an elastic material 3—or the strip 20—so as to be pressed and held against the knee.

The effect of the straps 30 is now described more specifically with reference to FIGS. 11 to 13.

Thanks to the bows 34 and branches 32, 33 which respectively pass below and above the knee, the straps 30 allow increasing the patella enclosing effect.

Furthermore, when the knee is bent (FIG. 13), the traction on the straps 30 increases, thereby increasing the pressure of the bows 30 on the holding device 1. The enclosing effect, and hence the patella centering effect, reaches its optimum when the knee is bent, which exactly corresponds to the situation where the risk of dislocation is at its maximum.

FIGS. 14 to 18 show how it is possible for the user to perfectly center the holding device 1 on his patella 100.

On FIG. 14, the holding device 1 is located too much to the left. Hence, by displacing it substantially horizontally to the right according to the arrow F1, it is possible to recenter it with respect to the patella 100 such as is illustrated on FIG. 16. Conversely, on FIG. 15, the holding device 1 is located too much to the right. Hence, by displacing it substantially horizontally to the left according to the arrow F2, it is possible to recenter it with respect to the patella 100. In order to displace the holding device 1, the user holds the tab 28 (or both tabs 28) of the positioning member 25 and pulls it in the appropriate direction.

The translational adjusting of the positioning member 25 may be performed at any given time of use and thus, without having to remove the orthosis 2.

To this end, the user has to detach the tabs 28 of the part made of an elastic material 3 so as to make them able to be held through the aperture 23 (FIG. 17). He/she may then pull on any of the tabs in the appropriate direction. Once the proper position is obtained, the tabs 28 are folded in order to be assembled again on the part made of an elastic material 3 and thereby secure this part 3 to the holding device 1 fixed on the positioning member 25 (FIG. 18).

Furthermore, according to a possible embodiment, and as is illustrated on FIGS. 19 to 23, the orthosis 2 may comprise, at least on one side of the leg equipped with the orthosis 2, and preferably on each side, substantially in the frontal plane when in the position of use, an upper bar 38 and a lower bar 39 articulated to each other substantially at the knee joint.

The bars 38, 39 are connected to each other by a plate 40 which is articulated to the upper bar 38 about an axis 41 orthogonal to the median plane—when in the position of use—and which is articulated to the lower bar 39 about an axis 42 orthogonal to the median plane. The bars 38, 39 and the plate 40 constitute a lateral strengthening member 45 which allows improving the hold of the orthosis 2. They may be made of a light alloy.

In addition, the adjacent ends of the bars 38, 39 are fitted with teeth 43 which, by cooperating, form a gear-type connection, and complementary posterior surfaces forming a stop 44.

The teeth 43 being arranged mainly at the posterior and lower parts of the adjacent ends of the bars 38, 39, the amplitude of the articulating movement between the bars 38, 39 is blocked, in the extension direction, at a flexion angle α of around 5°. Thus, it is avoided that the lower member is placed in a hyper-flexing position of the knee, as is illustrated on FIG. 19, this behavior contributing to the sub- dislocation of the patella.

Furthermore, as is illustrated on FIG. 23, the flexion of the knee is limited by the stop 44 to an amplitude β, for example close to 25°.

In practice, the bars 38, 39 and the plate 40 are mounted, on either side of the knee in the frontal plane when in the position of use, inside sheaths 46 arranged on the part made of an elastic material 3. The sheaths 46 may be provided on the strip 20 on either side of the aperture 23, as can be seen in particular on FIGS. 10 and 12. The sheaths 46 may have a surface that allows the hooking of a hook-and-loop fastening fabric, and thereby allows hooking of the tabs 28 of the positioning member 25 and the free ends of the branches 32, 33 of the straps 30.

Hooking the free ends of the branches 32, 33 of the straps 30 on the sheaths 46 receiving the bars 38, 39, allows improving the enclosing of the patella. In fact, when the knee is bent, the lateral strengthening members 45 are folded by relative pivoting of the bars 38, 39, the bars 38, 39 thereby exerting a traction on the straps 30 translated by an improved pressing of the holding device 1 against the knee.

Thus, the invention brings a determining improvement to the prior technique, by providing a holding device which allows for an excellent enclosing of the patella, at rest, and even more when bending.

It goes without saying that the invention is not limited to the aforementioned embodiment by way of example, but encompasses all technical equivalents and variants of the means described as well as their combinations.

1-16. (canceled)

17. A device for holding the patella intended to be placed against the knee of a person, comprising:
a substantially annular body having a central opening arranged to receive the patella and having a substantially oval edge;
a plurality of support members distinct from one another, protruding outwards from the periphery of the body, two adjacent support members being separated by a slit in an unconstrained state of the holding device;
the holding device being substantially planar along a plane when in the unconstrained state, and substantially non-deformable in the plane, and the holding device arranged to be deformed into a convex position forming the shape of the knee when bending, a convex position in which the support members are substantially contiguous, the holding device then substantially forming a cup enveloping the knee.

18. The holding device according to claim 17, wherein the support members are distributed substantially regularly over substantially the entire periphery of the body.

19. The holding device according to claim 17, wherein the number N of support members is such that 5≦N≦10.

20. The holding device according to claim 17, wherein the holding device has a plane of symmetry which, in position of use and when the person equipped with said holding device is standing, is substantially parallel with the median plane of the person.

21. The holding device according to claim 17, wherein the holding device comprises a lower support member arranged to be able to press against the anterior tibial tuberosity of the person equipped with said holding device.

22. The holding device according to claim 17, wherein the holding device includes a substantially planar external face, in the unconstrained state, and in that the support members have a face turned inwards arranged towards the person equipped with the holding device in the position of use and being convex.

23. The holding device according to claim 17, wherein the holding device is fixed on a positioning member arranged to be assembled, removably and displacably, on a part made of an elastic material intended to surround the knee.

24. A knee orthosis, wherein the knee orthosis comprises a holding device according to claim 17, and a part made of an elastic material intended to surround the knee and to press the holding device against the knee.

25. The orthosis according to claim 24, wherein the part made of an elastic material comprises a strip having lateral extreme parts provided with complementary hooking means, so that the strip may be closed and hooked around itself in the anterior part of the knee.

26. The orthosis according to claim 24, wherein the orthosis further comprises a positioning member on which the holding device is fixed, the positioning member being designed so as to be held by the user when the orthosis is in the position of use and to be displaced with respect to the part made of an elastic material in order to properly position the holding device around the patella, the positioning member further including removable assembly means on the part made of an elastic material in the appropriate position.

27. The orthosis according to claim 26, wherein the positioning member has a central portion on the internal face of which the holding device is fixed, as well as two lateral gripping tabs each of which being capable of passing through a lateral lumen arranged in the part made of an elastic material and which are fitted with means for assembly with said part made of an elastic material.

28. The orthosis according to claims 24, wherein the orthosis further comprises at least one V-shaped strap which has:
a tip fixed to the part made of an elastic material on a first side of the knee, the tip being directed opposite the knee;
a branch extending above the knee and another branch extending below the knee, the free end of the branches being removably fixed on the part made of an elastic material.

29. The orthosis according to claim 24, wherein it further comprises two V-shaped straps which, when in the position of use, are disposed in a substantially symmetrical manner on either side of the knee, a strap having:
a tip fixed to the part made of an elastic material on a first side of the knee, the tip being directed opposite the knee;
a branch extending above the knee and another branch extending below the knee, the free end of the branches being removably fixed on the part made of an elastic material.

30. The orthosis according to claim 29, wherein the holding device comprises a lower support member arranged to be able to press against the anterior tibial tuberosity of the person equipped with said holding device, and in that the two straps are arranged so that, when in the position of use, they intersect, below the knee, substantially on the lower support member of the holding device.

31. The orthosis according to claim 28, wherein at least one strap comprises, in the vicinity of its tip, a bow the concavity of which is directed towards the knee, when in the position of use.

32. The orthosis according to claim 24, wherein the orthosis comprises, at least on one side of the leg equipped with the orthosis, substantially in the frontal plane when in the position of use, two bars articulated together substantially at the articulation of the knee, the amplitude of the articulation movement being blocked, in the direction of the extension, at a flexion angle of the order of 5°.
